(19) United States
(12) Patent Application Publication
(10) Pub. No.: US 2024/0002316 A1
(43) Pub. Date: Jan. 4, 2024

(54) A PROCESS FOR PRODUCING ALPHA-OLEFINS

(71) Applicant: SHELL OIL COMPANY, Houston, TX (US)

(72) Inventors: Valerie Anne WILLIAMS, Monaca, PA (US); Cornelius Mark BOLINGER, Sugar land, TX (US); Brent Carl NORRIS, Houston, TX (US)

(21) Appl. No.: 18/253,057

(22) PCT Filed: Dec. 14, 2021
(86) PCT No.: PCT/US2021/063276

§ 371 Date: May 16, 2023

Related U.S. Application Data
(60) Provisional application No. 63/125,783, filed on Dec. 15, 2020.

(51) Int. Cl. C07C 2/34; B01J 31/40; B01J 31/14

(52) U.S. Cl. CPC C07C 2/34; B01J 31/403; C07C 2531/14; B01J 2231/20; B01J 31/143

(57) ABSTRACT

The invention provides a process for producing alpha-olefins comprising: a) contacting an ethylene feed with an oligomerization catalyst system, the catalyst system comprising a metal-ligand catalyst and a co-catalyst, in an oligomerization reaction zone under oligomerization conditions to produce a product stream comprising alpha-olefins; b) withdrawing the product stream from the oligomerization reaction zone wherein the product stream further comprises oligomerization catalyst system; c) contacting the product stream with a catalyst deactivating agent to form a deactivated product stream that contains deactivated catalyst components; and d) heating the deactivated product stream to separate one or more components from the deactivated product stream.

### FIELD OF THE INVENTION

The invention relates to a process for producing alpha-olefins and deactivating the catalyst used in that process.

### BACKGROUND

The oligomerization of olefins, such as ethylene, produces butene, hexene, octene, and other valuable linear alpha olefins. Linear alpha olefins are a valuable comonomer for linear low-density polyethylene and high-density polyethylene. Such olefins are also valuable as a chemical intermediate in the production of plasticizer alcohols, fatty acids, detergent alcohols, polyalphaolefins, oil field drilling fluids, lubricant oil additives, linear alkylbenzenes, alkenylsuccinic anhydrides, alkyldimethylamines, dialkylmethylamines, alpha-olefin sulfonates, internal olefin sulfonates, chlorinated olefins, linear mercaptans, aluminum alkyls, alkyldiphenylether disulfonates, and other chemicals.

U.S. Pat. No. 6,683,187 describes a bis(arylimino)pyridine ligand, catalyst precursors and catalyst systems derived from this ligand for ethylene oligomerization to form linear alpha olefins. The patent teaches the production of linear alpha olefins with a Schulz-Flory oligomerization product distribution. In such a process, a wide range of oligomers are produced, and the fraction of each olefin can be determined by calculation on the basis of the K-factor. The K-factor is the molar ratio of (Cₙ+2)/Cₙ, where n is the number of carbons in the linear alpha olefin product.

It would be advantageous to develop an improved process that would provide an oligomerization product distribution having a desired K-factor and product quality. The catalyst used in the process can produce undesired byproducts if it is still active during the downstream processing steps of the product stream.

### SUMMARY OF THE INVENTION

The invention provides a process for producing alpha-olefins comprising: a) contacting an ethylene feed with an oligomerization catalyst system, the catalyst system comprising a metal-ligand catalyst and a co-catalyst, in an oligomerization reaction zone under oligomerization conditions to produce a product stream comprising alpha-olefins; b) withdrawing the product stream from the oligomerization reaction zone wherein the product stream further comprises oligomerization catalyst system; c) contacting the product stream with a catalyst deactivating agent to form a deactivated product stream that contains deactivated catalyst components; and d) heating the deactivated product stream to separate one or more components from the deactivated product stream.

### BRIEF DESCRIPTION OF THE DRAWINGS

FIG. 1 depicts the results of Example 1.

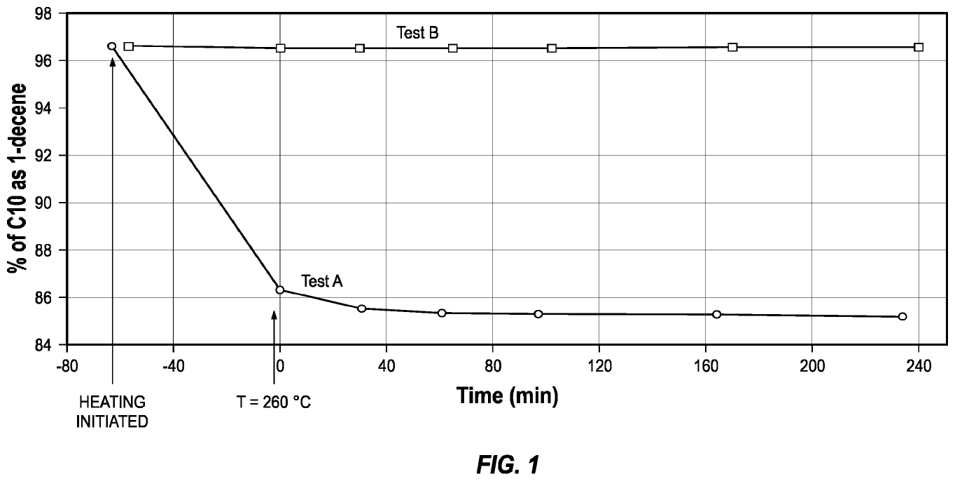

FIG. 2 depicts the results of Example 2.

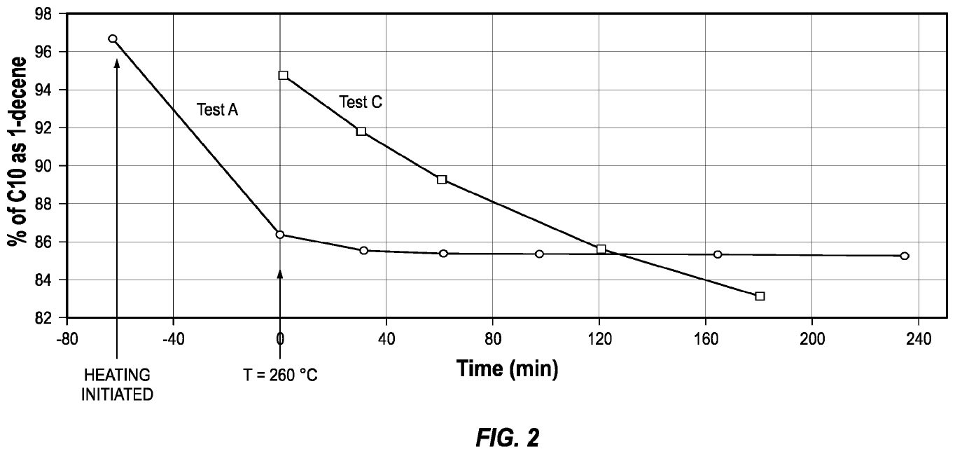

### DETAILED DESCRIPTION

The process comprises converting an olefin feed into a higher oligomer product stream by contacting the feed with an oligomerization catalyst system and a co-catalyst in an oligomerization reaction zone under oligomerization conditions. In one embodiment, an ethylene feed may be contacted with an iron-ligand complex and modified methyl aluminoxane under oligomerization conditions to produce a product slate of alpha olefins having a specific k-factor.

### Olefin Feed

The olefin feed to the process comprises ethylene. The feed may also comprise olefins having from 3 to 8 carbon atoms. The ethylene may be pretreated to remove impurities, especially impurities that impact the reaction, product quality or damage the catalyst. In one embodiment, the ethylene may be dried to remove water. In another embodiment, the ethylene may be treated to reduce the oxygen content of the ethylene. Any pretreatment method known to one of ordinary skill in the art can be used to pretreat the feed.

### Oligomerization Catalyst

The oligomerization catalyst system may comprise one or more oligomerization catalysts as described further herein. The oligomerization catalyst is a metal-ligand complex that is effective for catalyzing an oligomerization process. The ligand may comprise a bis(arylimino)pyridine compound, a bis(alkylimino)pyridine compound or a mixed aryl-alkyl iminopyridine compound.

### Ligand

In one embodiment, the ligand comprises a pyridine bis(imine) group. The ligand may be a bis(arylimino)pyridine compound having the structure of Formula I.

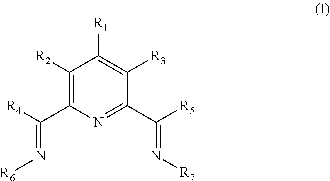

R₁, R₂ and R₃ are each independently hydrogen, optionally substituted hydrocarbyl, hydroxo, cyano or an inert functional group. R₄ and R₅ are each independently hydrogen, optionally substituted hydrocarbyl, hydroxo, cyano or an inert functional group. R₆ and R₇ are each independently an aryl group as shown in Formula II. The two aryl groups (R₆ and R₇) on one ligand may be the same or different.

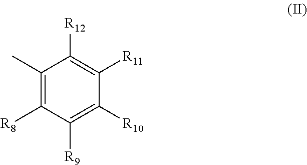

R₈, R₉, R₁₀, R₁₁, R₁₂ are each independently hydrogen, optionally substituted hydrocarbyl, hydroxo, cyano, an inert functional group, fluorine, or chlorine. Any two of R₁-R₃, and R₉-R₁₁ vicinal to one another taken together may form a ring. R₁₂ may be taken together with R₁₁, R₄ or R₅ to form a ring. R₂ and R₄ or R₃ and R₅ may be taken together to form a ring.

A hydrocarbyl group is a group containing only carbon and hydrogen. The number of carbon atoms in this group is preferably in the range of from 1 to 30.

An optionally substituted hydrocarbyl is a hydrocarbyl group that optionally contains one or more “inert” heteroatom-containing functional groups. Inert means that the functional groups do not interfere to any substantial degree with the oligomerization process. Examples of these inert groups include fluoride, chloride, iodide, stannanes, ethers, hydroxides, alkoxides and amines with adequate steric shielding. The optionally substituted hydrocarbyl group may include primary, secondary and tertiary carbon atoms groups.

Primary carbon atom groups are a —CH₂—R group wherein R may be hydrogen, an optionally substituted hydrocarbyl or an inert functional group. Examples of primary carbon atom groups include —CH₃, —C₂H₅, —CH₂Cl, —CH₂OCH₃, —CH₂N(C₂H₅)₂, and —CH₂Ph. Secondary carbon atom groups are a —CH—R₂ or —CH(R)(R′) group wherein R and R′ may be optionally substituted hydrocarbyl or an inert functional group. Examples of secondary carbon atom groups include —CH(CH₃)₂, —CHCl₂, —CHPh₂, —CH(CH₃)(OCH₃), —CH═CH₂, and cyclohexyl. Tertiary carbon atom groups are a —C—(R)(R′)(R″) group wherein R, R′, and R″ may be optionally substituted hydrocarbyl or an inert functional group. Examples of tertiary carbon atom groups include —C(CH₃)₃, —CCl₃, —C≡CPh, 1-Adamantyl, and —C(CH₃)₂(OCH₃)

An inert functional group is a group other than optionally substituted hydrocarbyl that is inert under the oligomerization conditions. Inert has the same meaning as provided above. Examples of inert functional groups include halide, ethers, and amines, in particular tertiary amines.

Substituent variations of R₁-R₅, R₈-R₁₂ and R₁₃-R₁₇ may be selected to enhance other properties of the ligand, for example, solubility in non-polar solvents. Several embodiments of possible oligomerization catalysts are further described below having the structure shown in Formula 3.

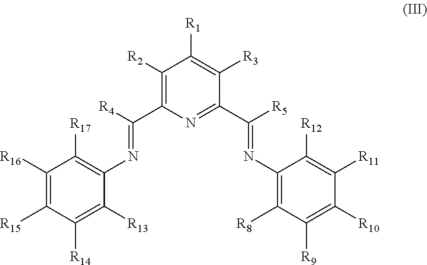

In one embodiment, a ligand of Formula III is provided wherein R₁-R₅, R₉-R₁₁ and R₁₄-R₁₆ are hydrogen; and R₈, R₁₂, R₁₃ and R₁₇ are fluorine.

In one embodiment, a ligand of Formula III is provided wherein R₁-R₅, R₈, R₁₀, R₁₂, R₁₄ and R₁₆ are hydrogen; R₁₃, R₁₅ and R₁₇ are methyl and R₉ and R₁₁ are tert-butyl.

In one embodiment, a ligand of Formula III is provided wherein R₁-R₅, R₈, R₁₂, R₁₄ and R₁₆ are hydrogen; R₁₃, R₁₅ and R₁₇ are methyl; R₉ and R₁₁ are phenyl and R₁₀ is an alkoxy.

In one embodiment, a ligand of Formula III is provided wherein R₁-R₅, R₈, R₁₀, R₁₁ and R₁₄-R₁₆ are hydrogen; R₉ and R₁₂ are methyl; and R₁₃ and R₁₇ are fluorine.

In one embodiment, a ligand of Formula III is provided wherein R₁-R₃, R₉-R₁₁ and R₁₄-R₁₆ are hydrogen; R₄ and R₅ are phenyl and R₈, R₁₂, R₁₃ and R₁₇ are fluorine.

In one embodiment, a ligand of Formula III is provided wherein R₁-R₅, R₈-R₉, R₁₁-R₁₂, R₁₃-R₁₄ and R₁₆-R₁₇ are hydrogen; and R₁₀ and R₁₅ are fluorine.

In one embodiment, a ligand of Formula III is provided wherein R₁-R₅, R₈, R₁₀, R₁₂, R₁₃, R₁₅ and R₁₇ are hydrogen; and R₉, R₁₁, R₁₄ and R₁₆ are fluorine.

In one embodiment, a ligand of Formula III is provided wherein R₁-R₅, R₉, R₁₁-R₁₂, R₁₄ and R₁₆-R₁₇ are hydrogen; and R₈, R₁₀, R₁₃ and R₁₅ are fluorine.

In one embodiment, a ligand of Formula III is provided wherein R₁-R₅, R₈-R₉, R₁₁-R₁₂, R₁₄ and R₁₆ are hydrogen; R₁₀ is tert-butyl; and R₁₃, R₁₅ and R₁₇ are methyl.

In one embodiment, a ligand of Formula III is provided wherein R₁-R₅, R₉-R₁₂, R₁₄ and R₁₆ are hydrogen; R₈ is fluorine; and R₁₃, R₁₅ and R₁₇ are methyl.

In one embodiment, a ligand of Formula III is provided wherein R₁-R₅, R₉-R₁₂, R₁₃, R₁₅ and R₁₇ are hydrogen; R₈ is tert-butyl; and R₁₄ and R₁₆ are methyl.

In one embodiment, a ligand of Formula III is provided wherein R₁-R₅, R₉-R₁₂, R₁₃-R₁₄ and R₁₆-R₁₇ are hydrogen; and R₈ and R₁₅ are tert-butyl.

In one embodiment, a ligand of Formula III is provided wherein R₁-R₅, R₈-R₁₀, R₁₃-R₁₄ and R₁₆-R₁₇ are hydrogen; R₁₅ is tert-butyl; and R₁₁ and R₁₂ are taken together to form an aryl group.

In one embodiment, a ligand of Formula III is provided wherein R₁-R₅, R₉-R₁₂, R₁₄-R₁₇ are hydrogen; and R₈ and R₁₃ are methyl.

In one embodiment, a ligand of Formula III is provided wherein R₁-R₅, R₈-R₉, R₁₁-R₁₂, R₁₄ and R₁₆ are hydrogen; R₁₀ is fluorine; and R₁₃, R₁₅ and R₁₇ are methyl.

In one embodiment, a ligand of Formula III is provided wherein R₁-R₅, R₈, R₁₀, R₁₂, R₁₄ and R₁₆ are hydrogen; R₉ and R₁₁ are fluorine; and R₁₃, R₁₅ and R₁₇ are methyl.

In one embodiment, a ligand of Formula III is provided wherein R₁-R₅, R₈-R₉, R₁₁-R₁₂, R₁₄ and R₁₆ are hydrogen; R₁₀ is an alkoxy; and R₁₃, R₁₅ and R₁₇ are methyl.

In one embodiment, a ligand of Formula III is provided wherein R₁-R₅, R₈-R₉, R₁₁-R₁₂, R₁₄ and R₁₆ are hydrogen; R₁₀ is a silyl ether; and R₁₃, R₁₅ and R₁₇ are methyl.

In one embodiment, a ligand of Formula III is provided wherein R₁-R₅, R₈, R₁₀, R₁₂, R₁₄-R₁₆ are hydrogen; R₉ and R₁₁ are methyl; and R₁₃ and R₁₇ are ethyl.

In one embodiment, a ligand of Formula III is provided wherein R₁-R₅, R₉-R₁₂, and R₁₄-R₁₇ are hydrogen; and R₈ and R₁₃ are ethyl.

In one embodiment, a ligand of Formula III is provided wherein R₁-R₅, R₉-R₁₁ and R₁₄-R₁₆ are hydrogen; and R₈, R₁₂, R₁₃ and R₁₇ are chlorine.

In one embodiment, a ligand of Formula III is provided wherein R₁-R₅, R₉, R₁₁, R₁₄ and R₁₆ are hydrogen; and R₈, R₁₀, R₁₂, R₁₃, R₁₅ and R₁₇ are methyl.

In one embodiment, a ligand of Formula III is provided wherein R₁-R₅, R₉-R₁₀, R₁₂, R₁₄-R₁₅ and R₁₇ are hydrogen; and R₈, R₁₁, R₁₃ and R₁₆ are methyl.

In one embodiment, a ligand of Formula III is provided wherein R₁-R₁₇ are hydrogen.

In one embodiment, a ligand of Formula III is provided wherein R₁-R₅, R₈, R₁₀, R₁₂, R₁₃, R₁₅ and R₁₇ are hydrogen; and R₉, R₁₁, R₁₄ and R₁₆ are tert-butyl.

In one embodiment, a ligand of Formula III is provided wherein R₁-R₅, R₈-R₁₂, R₁₄ and R₁₆ are hydrogen; and R₁₃, R₁₅ and R₁₇ are methyl.

In one embodiment, a ligand of Formula III is provided wherein R₁-R₅, R₉, R₁₁-R₁₂, R₁₄ and R₁₆ are hydrogen; R₈ and R₁₀ are fluorine; and R₁₃, R₁₅ and R₁₇ are methyl.

In one embodiment, a ligand of Formula III is provided wherein R₁-R₅, R₉, R₁₁-R₁₂, R₁₄ and R₁₆-R₁₇ are hydrogen; and R₈, R₁₀, R₁₃ and R₁₅ are methyl.

In one embodiment, a ligand of Formula III is provided wherein R₁-R₅, R₉-R₁₁ and R₁₄-R₁₆ are hydrogen; R₈ and R₁₂ are chlorine; and R₁₃ and R₁₇ are fluorine.

In one embodiment, a ligand of Formula III is provided wherein R₁-R₅, R₈, R₁₀, R₁₂, R₁₄ and R₁₆ are hydrogen; and R₉, R₁₁, R₁₃, R₁₅ and R₁₇ are methyl.

In one embodiment, a ligand of Formula III is provided wherein R₁-R₅, R₉-R₁₁ and R₁₃-R₁₄ and R₁₆-R₁₇ are hydrogen; R₈ and R₁₂ are chlorine; and R₁₅ is tert-butyl.

In one embodiment, a ligand of Formula III is provided wherein R₁-R₅, R₉-R₁₁ and R₁₃-R₁₇ are hydrogen; and R₈ and R₁₂ are chlorine.

In one embodiment, a ligand of Formula III is provided wherein R₁-R₅, R₉-R₁₂, and R₁₄-R₁₇ are hydrogen; and R₈ and R₁₃ are chlorine.

In one embodiment, a ligand of Formula III is provided wherein R₁-R₅, R₉, R₁₁-R₁₂, R₁₄ and R₁₆-R₁₇ are hydrogen; and R₈, R₁₀, R₁₃ and R₁₅ are chlorine.

In one embodiment, a ligand of Formula III is provided wherein R₁-R₅, R₉, R₁₁-R₁₂, and R₁₄, and R₁₆-R₁₇ are hydrogen; R₁₀ and R₁₅ are methyl; and R₈ and R₁₃ are chlorine.

In one embodiment, a ligand of Formula III is provided wherein R₁-R₅, R₉-R₁₁ and R₁₃-R₁₄ and R₁₆-R₁₇ are hydrogen; R₁₅ is fluorine; and R₈ and R₁₂ are chlorine.

In one embodiment, a ligand of Formula III is provided wherein R₁-R₅, R₈-R₉, R₁₁-R₁₂, R₁₄-R₁₅ and R₁₇ are hydrogen; R₁₀ is tert-butyl; and R₁₃ and R₁₆ are methyl.

In one embodiment, a ligand of Formula III is provided wherein R₁-R₅, R₉-R₁₁, R₁₄ and R₁₆ are hydrogen; R₈ and R₁₂ are fluorine; and R₁₃, R₁₅ and R₁₇ are methyl.

In one embodiment, a ligand of Formula III is provided wherein R₁-R₅, R₉-R₁₀, R₁₂, R₁₄-R₁₅ and R₁₇ are hydrogen; R₈ and R₁₃ are methyl; and R₁₁ and R₁₆ are isopropyl.

In one embodiment, a ligand of Formula III is provided wherein R₁-R₅, R₉-R₁₂ and R₁₄-R₁₆ are hydrogen; Re is ethyl; and R₁₃ and R₁₇ are fluorine.

In one embodiment, a ligand of Formula III is provided wherein R₂-R₅, R₉-R₁₀, R₁₂, R₁₄-R₁₅ and R₁₇ are hydrogen; R₁ is methoxy; and R₈, R₁₁, R₁₃ and R₁₆ are methyl.

In one embodiment, a ligand of Formula III is provided wherein R₂-R₅, R₈-R₁₂, R₁₄ and R₁₆ are hydrogen; R₁ is methoxy; and R₁₃, R₁₅ and R₁₇ are methyl.

In one embodiment, a ligand of Formula III is provided wherein R₂-R₅, R₉-R₁₂, and R₁₄-R₁₇ are hydrogen; R₁ is methoxy; and R₈ and R₁₃ are ethyl.

In one embodiment, a ligand of Formula III is provided wherein R₂-R₅, R₉, R₁₁-R₁₂, R₁₄ and R₁₆-R₁₇ are hydrogen; R₁ is tert-butyl; and R₈, R₁₀, R₁₃ and R₁₅ are methyl.

In one embodiment, a ligand of Formula III is provided wherein R₂-R₅, R₈-R₁₂, R₁₄ and R₁₆ are hydrogen; R₁ is tert-butyl; and R₁₃, R₁₅ and R₁₇ are methyl.

In one embodiment, a ligand of Formula III is provided wherein R₂-R₅, R₉, R₁₁, R₁₄ and R₁₆ are hydrogen; R₁ is methoxy; and R₈, R₁₀, R₁₂, R₁₃, R₁₅ and R₁₇ are methyl.

In one embodiment, a ligand of Formula III is provided wherein R₂-R₅, R₉, R₁₁, R₁₄ and R₁₆ are hydrogen; R₁ is alkoxy; and R₈, R₁₀, R₁₂, R₁₃, R₁₅ and R₁₇ are methyl.

In one embodiment, a ligand of Formula III is provided wherein R₂-R₅, R₉, R₁₁, R₁₄ and R₁₆ are hydrogen; R₁ is tert-butyl; and R₈, R₁₀, R₁₂, R₁₃, R₁₅ and R₁₇ are methyl.

In another embodiment, the ligand may be a compound having the structure of Formula I, wherein one of R₆ and R₇ is aryl as shown in Formula II and one of R₆ and R₇ is pyridyl as shown in Formula IV. In another embodiment, R₆ and R₇ may be pyrrolyl.

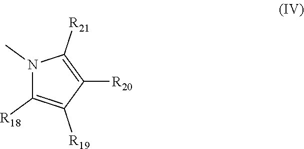

R₁, R₂ and R₃ are each independently hydrogen, optionally substituted hydrocarbyl, hydroxo, cyano or an inert functional group. R₄ and R₅ are each independently hydrogen, optionally substituted hydrocarbyl, hydroxo, cyano or an inert functional group. R₈-R₁₂ and R₁₈-R₂₁ are each independently hydrogen, optionally substituted hydrocarbyl, hydroxo, cyano, an inert functional group, fluorine, or chlorine. Any two of R₁-R₃, and R₉-R₁₁ vicinal to one another taken together may form a ring. R₁₂ may be taken together with R₁₁, R₄ or R₅ to form a ring. R₂ and R₄ or R₃ and R₅ may be taken together to form a ring.

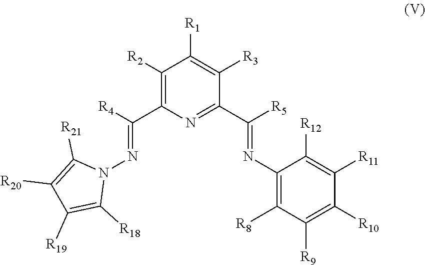

In one embodiment, a ligand of Formula V is provided wherein R₁-R₅, R₉, R₁₁ and R₁₈-R₂₁ are hydrogen; and R₈, R₁₀, and R₁₂ are methyl.

In one embodiment, a ligand of Formula V is provided wherein R₁-R₅, R₉-R₁₁ and R₁₈-R₂₁ are hydrogen; and R₈ and R₁₂ are ethyl.

In another embodiment, the ligand may be a compound having the structure of Formula I, wherein one of R₆ and R₇ is aryl as shown in Formula II and one of R₆ and R₇ is cyclohexyl as shown in Formula VI. In another embodiment, R₆ and R₇ may be cyclohexyl.

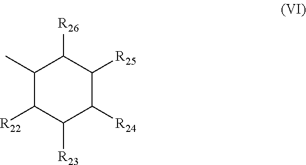

R₁, R₂ and R₃ are each independently hydrogen, optionally substituted hydrocarbyl, hydroxo, cyano or an inert functional group. R₄ and R₅ are each independently hydrogen, optionally substituted hydrocarbyl, hydroxo, cyano or an inert functional group. R₈-R₁₂ and R₂₂-R₂₆ are each independently hydrogen, optionally substituted hydrocarbyl, hydroxo, cyano, an inert functional group, fluorine, or chlorine. Any two of R₁-R₃, and R₉-R₁₁ vicinal to one another taken together may form a ring. R₁₂ may be taken together with R₁₁, R₄ or R₅ to form a ring. R₂ and R₄ or R₃ and R₅ may be taken together to form a ring.

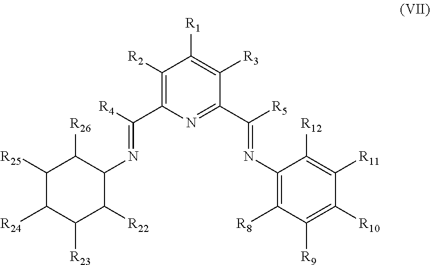

In one embodiment, a ligand of Formula VII is provided wherein R₁-R₅, R₉, R₁₁ and R₂₂-R₂₆ are hydrogen; and R₈, R₁₀, and R₁₂ are methyl.

In another embodiment, R₆ and R₇ may be adamantyl or another cycloalkane.

In another embodiment, the ligand may be a compound having the structure of Formula I, wherein one of R₆ and R₇ is aryl as shown in Formula II and one of R₆ and R₇ is ferrocenyl as shown in Formula VIII. In another embodiment, R₆ and R₇ may be ferrocenyl.

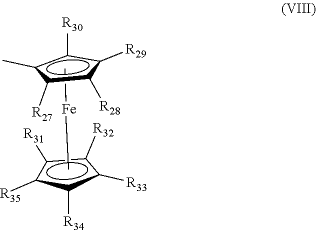

R₁, R₂ and R₃ are each independently hydrogen, optionally substituted hydrocarbyl, hydroxo, cyano or an inert functional group. R₄ and R₅ are each independently hydrogen, optionally substituted hydrocarbyl, hydroxo, cyano or an inert functional group. R₈-R₁₂ and R₂₇-R₃₅ are each independently hydrogen, optionally substituted hydrocarbyl, hydroxo, cyano, an inert functional group, fluorine, or chlorine. Any two of R₁-R₃, and R₉-R₁₁ vicinal to one another taken together may form a ring. R₁₂ may be taken together with R₁₁, R₄ or R₅ to form a ring. R₂ and R₄ or R₃ and R₅ may be taken together to form a ring.

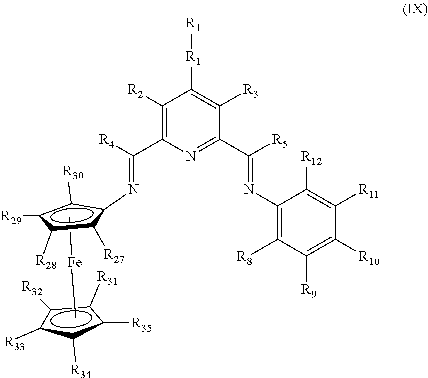

In one embodiment, a ligand of Formula IX is provided wherein R₁-R₅, R₉, R₁₁ and R₂₇-R₃₅ are hydrogen; and R₈, R₁₀, and R₁₂ are methyl.

In one embodiment, a ligand of Formula IX is provided wherein R₁-R₅, R₉-R₁₁, and R₂₇-R₃₅ are hydrogen; and R₈ and R₁₂ are ethyl.

In another embodiment, the ligand may be a bis(alkylamino)pyridine. The alkyl group may have from 1 to 50 carbon atoms. The alkyl group may be a primary, secondary, or tertiary alkyl group. The alkyl group may be selected from the group consisting of methyl, ethyl, propyl, isopropyl, butyl, sec-butyl, isobutyl, and tert-butyl. The alkyl group may be selected from any n-alkyl or structural isomer of an n-alkyl having 5 or more carbon atoms, e.g., n-pentyl; 2-methyl-butyl; and 2,2-dimethylpropyl.

In another embodiment, the ligand may be an alkyl-alkyl iminopyridine, where the two alkyl groups are different. Any of the alkyl groups described above as being suitable for a bis(alkylamino)pyridine are also suitable for this alkyl-alkyl iminopyridine.

In another embodiment, the ligand may be an aryl alkyl iminopyridine. The aryl group may be of a similar nature to any of the aryl groups described with respect to the bis(arylimino)pyridine compound and the alkyl group may be of a similar nature to any of the alkyl groups described with respect to the bis(alkylamino)pyridine compound.

In addition to the ligand structures described hereinabove, any structure that combines features of any two or more of these ligands can be a suitable ligand for this process. Further, the oligomerization catalyst system may comprise a combination of one or more of any of the described oligomerizations catalysts.

The ligand feedstock may contain between 0 and 10 wt. % bisimine pyridine impurity, preferably 0-1 wt. % bisimine pyridine impurity, most preferably 0-0.1 wt. % bisimine pyridine impurity. This impurity is believed to cause the formation of polymers in the reactor, so it is preferable to limit the amount of this impurity that is present in the catalyst system.

In one embodiment, the bisimine pyridine impurity is a ligand of Formula II in which three of R₈, R₁₂, R₁₃, and R₁₇ are each independently optionally substituted hydrocarbyl.

In one embodiment, the bisimine pyridine impurity is a ligand of Formula II in which all four of R₈, R₁₂, R₁₃, and R₁₇ are each independently optionally substituted hydrocarbyl.

### Metal

The metal may be a transition metal, and the metal is preferably present as a compound having the formula MXₙ, where M is the metal, X is a monoanion and n represents the number of monoanions (and the oxidation state of the metal).

The metal can comprise any Group 4-10 transition metal. The metal can be selected from the group consisting of titanium, zirconium, hafnium, vanadium, niobium, tantalum, chromium, molybdenum, tungsten, manganese, iron, cobalt, nickel, palladium, platinum, ruthenium and rhodium. In one embodiment, the metal is cobalt or iron. In a preferred embodiment, the metal is iron. The metal of the metal compound can have any positive formal oxidation state of from 2 to 6 and is preferably 2 or 3.

The monoanion may comprise a halide, a carboxylate, a β-diketonate, a hydrocarboxide, an optionally substituted hydrocarbyl, an amide or a hydride. The hydrocarboxide may be an alkoxide, an aryloxide or an aralkoxide. The halide may be fluorine, chlorine, bromine or iodine.

The carboxylate may be any C₁ to C₂₀ carboxylate. The carboxylate may be acetate, a propionate, a butyrate, a pentanoate, a hexanoate, a heptanoate, an octanoate, a nonanoate, a decanoate, an undecanoate, or a dodecanoate. In addition, the carboxylate may be 2-ethylhexanoate or trifluoroacetate.

The β-diketonate may be any C₁ to C₂₀ β-diketonate. The β-diketonate may be acetylacetonate, hexafluoroacetylacetonate, or benzoylacetonate.

The hydrocarboxide may be any C₁ to C₂₀ hydrocarboxide. The hydrocarboxide may be a C₁ to C₂₀ alkoxide, or a C₆ to C₂₀ aryloxide. The alkoxide may be methoxide, ethoxide, a propoxide (e.g., iso-propoxide) or a butoxide (e.g., tert-butoxide). The aryloxide may be phenoxide

Generally, the number of monoanions equals the formal oxidation state of the metal atom.

Preferred embodiments of metal compounds include iron acetylacetonate, iron chloride, and iron bis(2-ethylhexanoate). In addition to the oligomerization catalyst, a co-catalyst is used in the oligomerization reaction.

### Co-Catalyst

The co-catalyst may be a compound that is capable of transferring an optionally substituted hydrocarbyl or hydride group to the metal atom of the catalyst and is also capable of abstracting an X⁻ group from the metal atom M. The co-catalyst may also be capable of serving as an electron transfer reagent or providing sterically hindered counterions for an active catalyst.

The co-catalyst may comprise two compounds, for example one compound that is capable of transferring an optionally substituted hydrocarbyl or hydride group to metal atom M and another compound that is capable of abstracting an X⁻ group from metal atom M. Suitable compounds for transferring an optionally substituted hydrocarbyl or hydride group to metal atom M include organoaluminum compounds, alkyl lithium compounds, Grignards, alkyl tin and alkyl zinc compounds. Suitable compounds for abstracting an X⁻ group from metal atom M include strong neutral Lewis acids such as SbF₅, BF₃ and Ar₃B wherein Ar is a strong electron-withdrawing aryl group such as C₆F₅ or 3,5-(CF₃)₂C₆H₃. A neutral Lewis acid donor molecule is a compound which may suitably act as a Lewis base, such as ethers, amines, sulfides and organic nitrites.

The co-catalyst is preferably an organoaluminum compound which may comprise an alkylaluminum compound, an aluminoxane or a combination thereof.

The alkylaluminum compound may be trialkylaluminum, an alkylaluminum halide, an alkylaluminum alkoxide or a combination thereof. The alkyl group of the alkylaluminum compound may be any C₁ to C₂₀ alkyl group. The alkyl group may be methyl, ethyl, propyl, butyl, pentyl, hexyl, heptyl or octyl. The alkyl group may be an iso-alkyl group.

The trialkylaluminum compound may comprise trimethylaluminum (TMA), triethylaluminum (TEA), tripropylaluminum, tributylaluminum, tripentylaluminum, trihexylaluminum, triheptylaluminum, trioctylaluminum or mixtures thereof. The trialkylaluminum compound may comprise tri-n-propylaluminum (TNPA), tri-n-butylaluminum (TNBA), tri-iso-butylaluminum (TIBA), tri-n-hexylaluminum, tri-n-octylaluminum (TNOA).

The halide group of the alkylaluminum halide may be chloride, bromide or iodide. The alkylaluminum halide may be diethylaluminum chloride, diethylaluminum bromide, ethylaluminum dichloride, ethylaluminum sesquichloride or mixtures thereof.

The alkoxide group of the alkylaluminum alkoxide may be any C₁ to C₂₀ alkoxy group. The alkoxy group may be methoxy, ethoxy, propoxy, butoxy, pentoxy, hexoxy, heptoxy or octoxy. The alkylaluminum alkoxide may be diethylaluminum ethoxide.

The aluminoxane compound may be methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentyl-aluminoxane, 2-pentyl-aluminoxane, 3-pentyl-aluminoxane, iso-pentyl-aluminoxane, neopentylaluminoxane, or mixtures thereof.

The preferred co-catalyst is modified methylaluminoxane. The synthesis of modified methylaluminoxane may be carried out in the presence of other trialkylaluminum compounds in addition to trimethylaluminum. The products incorporate both methyl and alkyl groups from the added trialkylaluminum and are referred to as modified methyl aluminoxanes, MMAO. The MMAO may be more soluble in nonpolar reaction media, more stable to storage, have enhanced performance as a cocatalyst, or any combination of these. The performance of the resulting MMAO may be superior to either of the trialkylaluminum starting materials or to simple mixtures of the two starting materials. The added trialkylaluminum may be triethylaluminum, triisobutylaluminum or triisooctylaluminum. In one embodiment, the co-catalyst is MMAO, wherein preferably about 25% of the methyl groups are replaced with iso-butyl groups.

In one embodiment, the co-catalyst may be formed in situ in the reactor by providing the appropriate precursors into the reactor.

### Solvent

One or more solvents may be used in the reaction. The solvent(s) may be used to dissolve or suspend the catalyst or the co-catalyst and/or keep the ethylene dissolved. The solvent may be any solvent that can modify the solubility of any of these components or of reaction products. Suitable solvents include hydrocarbons, for example, alkanes, alkenes, cycloalkanes, and aromatics. Different solvents may be used in the process, for example, one solvent can be used for the catalyst and another for the co-catalyst. It is preferred for the solvent to have a boiling point that is not substantially similar to the boiling point of any of the alpha olefin products as this will make the product separation step more difficult.

### Aromatics

Aromatic solvents can be any solvent that contains an aromatic hydrocarbon, preferably having a carbon number of 6 to 20. These solvents may include pure aromatics, or mixtures of pure aromatics, isomers as well as heavier solvents, for example C₉ and C₁₀ solvents. Suitable aromatic solvents include benzene, toluene, xylene (including ortho-xylene, meta-xylene, para-xylene and mixtures thereof) and ethylbenzene.

### Alkanes

Alkane solvents may be any solvent that contains an alkyl hydrocarbon. These solvents may include straight chain alkanes and branched or iso-alkanes having from 3 to 20 carbon atoms and mixtures of these alkanes. The alkanes may be cycloalkanes. Suitable solvents include propane, iso-butane, n-butane, butane (n-butane or a mixture of linear and branched C₄ acyclic alkanes), pentane (n-pentane or a mixture of linear and branched acyclic alkanes), hexane (n-hexane or a mixture of linear and branched C₆ acyclic alkanes), heptane (n-heptane or a mixture of linear and branched C₇ acyclic alkanes), octane (n-octane or a mixture of linear and branched C₈ acyclic alkanes) and isooctane. Suitable solvents also include cyclohexane and methylcyclohexane. In one embodiment, the solvent comprises C₆, C₇ and C₈ alkanes, that may include linear, branched and iso-alkanes.

### Catalyst System

The catalyst system may be formed by mixing together the ligand, the metal, the co-catalyst and optional additional compounds in a solvent. The feed may be present in this step.

In one embodiment, the catalyst system may be prepared by contacting the metal or metal compound with the ligand to form a catalyst precursor mixture and then contacting the catalyst precursor mixture with the co-catalyst in the reactor to form the catalyst system.

In some embodiments, the catalyst system may be prepared outside of the reactor vessel and fed into the reactor vessel. In other embodiments, the catalyst system may be formed in the reactor vessel by passing each of the components of the catalyst system separately into the reactor. In other embodiments, one or more catalyst precursors may be formed by combining at least two components outside of the reactor and then passing the one or more catalyst precursors into the reactor to form the catalyst system.

### Reaction Conditions

The oligomerization reaction is a reaction that converts the olefin feed in the presence of an oligomerization catalyst and a co-catalyst into a higher oligomer product stream.

### Temperature

The oligomerization reaction may be conducted over a range of temperatures of from −100° C. to 300° C., preferably in the range of from 0° C. to 200° C., and more preferably in the range of from 50° C. to 150° C.

### Pressure

The oligomerization reaction may be conducted at a pressure of from 0.01 to 15 MPa and more preferably from 1 to 10 MPa.

The optimum conditions of temperature and pressure used for a specific catalyst system, to maximize the yield of oligomer, and to minimize the impact of competing reactions, for example dimerization and polymerization can be determined by one of ordinary skill in the art. The temperature and pressure are selected to yield a product slate with a K-factor in the range of from 0.40 to 0.90, preferably in the range of from 0.45 to 0.80, more preferably in the range of from 0.5 to 0.7.

### Residence Time

Residence times in the reactor of from 3 to 60 min have been found to be suitable, depending on the activity of the catalyst. In one embodiment, the reaction is carried out in the absence of air and moisture.

### Gas Phase, Liquid Phase or Mixed Gas-Liquid Phase

The oligomerization reaction can be carried out in the liquid phase or mixed gas-liquid phase, depending on the volatility of the feed and product olefins at the reaction conditions.

### Reactor Type

The oligomerization reaction may be carried out in a conventional fashion. It may be carried out in a stirred tank reactor, wherein solvent, olefin and catalyst or catalyst precursors are added continuously to a stirred tank and solvent, product, catalyst, and unused reactant are removed from the stirred tank with the product separated and the unused reactant recycled back to the stirred tank.

In another embodiment, the oligomerization reaction may be carried out in a batch reactor, wherein the catalyst precursors and reactant olefin are charged to an autoclave or other vessel and after being reacted for an appropriate time, product is separated from the reaction mixture by conventional means, for example, distillation.

In another embodiment, the oligomerization reaction may be carried out in a gas lift reactor. This type of reactor has two vertical sections (a riser section and a downcomer section) and a gas separator at the top. The gas feed (ethylene) is injected at the bottom of the riser section to drive circulation around the loop (up the riser section and down the downcomer section).

In another embodiment, the oligomerization reaction may be carried out in a pump loop reactor. This type of reactor has two vertical sections, and it uses a pump to drive circulation around the loop. A pump loop reactor can be operated at a higher circulation rate than a gas lift reactor.

In another embodiment, the oligomerization reaction may be carried out in a once-through reactor. This type of reactor feeds the catalyst, co-catalyst, solvent and ethylene to the inlet of the reactor and/or along the reactor length and the product is collected at the reactor outlet. One example of this type of reactor is a plug flow reactor.

### Catalyst Deactivation

The higher oligomers produced in the oligomerization reaction contains catalyst from the reaction step. To stop further reactions that can produce byproducts and other undesired components, it is important to deactivate the catalyst downstream from the reactor. The catalyst system used for this oligomerization can convert nonconjugated dienes to conjugated dienes at the higher temperatures present in the downstream separation columns, specifically in the reboilers. These conjugated dienes are poisons to polyethylene catalyst, so it is important to prevent this conversion to conjugated dienes that would render the alpha-olefins off-spec. In addition to the conversion of dienes, the desired alpha-olefin products are also isomerized at higher temperatures in the presence of catalyst and cocatalyst that has not been deactivated.

In one embodiment, the alpha-olefins produced in the oligomerization reaction zone are contacted with a catalyst deactivating agent before the product stream is heated to separate the product stream. This separation is typically conducted by distillation, so it is important to deactivate the catalyst before the product stream is heated in the distillation section.

In another embodiment, the temperature of the deactivated product stream is no more than 10° C. higher than the temperature of the product stream exiting the reaction zone. In a further embodiment, the temperature of the product stream is less than 260° C., preferably less than 204° C., more preferably less than 150° C. and most preferably less than 135° C. before it has been contacted with a catalyst deactivating agent.

In one embodiment, the catalyst is deactivated by addition of an acidic species having a pKₐ(aq) of 25 or less, preferably of 20 or less. The deactivated catalyst can then be removed by aqueous washing in a liquid/liquid extractor. In one embodiment, the catalyst deactivating agent comprises a carboxylic acid. In a preferred embodiment, the catalyst deactivating agent is 2-ethylhexanoic acid.

In another embodiment, the catalyst deactivating agent comprises one or more esters. In a preferred embodiment, the catalyst deactivating agent comprises methyl acetoacetate.

It is preferred for the catalyst deactivating agent to remain in the heaviest product fraction as the products are separated into various products. The catalyst deactivating agent preferably has a boiling point of at least 170° C. and preferably at least 200° C. The catalyst deactivating agent may have a boiling point in the range of from 180 to 250° C.

### Product Separation

The resulting alpha-olefins have a chain length of from 4 to 100 carbon atoms, preferably 4 to 30 carbon atoms and most preferably 4 to 20 carbon atoms. The alpha-olefins are even-numbered alpha-olefins.

The product olefins can be recovered by distillation or other separation techniques depending on the intended use of the products. The solvent(s) used in the reaction preferably have a boiling point that is different from the boiling point of any of the alpha-olefin products to make the separation easier.

In one embodiment, the distillation steps comprise columns for separating ethylene and the main linear alpha olefin products, for example, butene, hexene, and octene.

The separation also comprises a step for removing the deactivated catalyst components. This separation may comprise containing the product stream or a portion of the product stream with an aqueous base. In one embodiment, the aqueous base comprises an alkali hydroxide, preferably potassium or sodium hydroxide. In one embodiment, this separation is conducted on the bottoms of a distillation column at the end of the distillation train (i.e., the heaviest stream). It is preferred to choose a catalyst deactivating agent that distributes to the aqueous layer in this step instead of distributing to the olefin layer (where it would remain as a product impurity).

### Product Qualities and Characteristics

The products produced by the process may be used in a number of applications. The olefins produced by this process may have improved qualities as compared to olefins produced by other processes. In one embodiment, the butene, hexene and/or octene produced may be used as a comonomer in making polyethylene. In one embodiment, the octene produced may be used to produce plasticizer alcohols. In one embodiment, the decene produced may be used to produce polyalphaolefins. In one embodiment, the dodecene and/or tetradecene produced may be used to produce alkylbenzene and/or detergent alcohols. In one embodiment, the hexadecene and/or octadecene produced may be used to produce alkenyl succinates and/or oilfield chemicals. In one embodiment, the C20+ products may be used to produce lubricant additives and/or waxes.

### Recycle

A portion of any unreacted ethylene that is removed from the reactor with the products may be recycled to the reactor. This ethylene may be recovered in the distillation steps used to separate the products. The ethylene may be combined with the fresh ethylene feed or it may be fed separately to the reactor.

A portion of any solvent used in the reaction may be recycled to the reactor. The solvent may be recovered in the distillation steps used to separate the products.

### EXAMPLES

### Example 1

Test A: MMAO (7 wt % Al in heptane) was added to a flask and diluted with a solution of 67 wt % 1-decene (C10 stream) in heptane so that the [Al]=500 ppmw. 3 molar equivalents of the deactivating agent (2-ethylhexanoic acid in this example) were slowly added to the mixture while stirring. After gas evolution was no longer observed, a solution of 0.25 wt % iron catalyst (iron duroct+Ligand A, 1:1.9 molar ratio) in heptane was added to the mixture. Ligand A is a ligand of Formula III wherein R₁-R₅, R₉, R₁₁-R₁₂, R₁₄ and R₁₆-R₁₇ are hydrogen; and R₈, R₁₀, R₁₃ and R₁₅ are methyl The mixture was transferred to a stainless-steel autoclave with a stir bar and sealed within the glovebox. The vessel was removed from the glovebox and heated to 260° C. for 2-4 hours. Periodically, aliquots were removed from the reaction vessel, cooled, and analyzed by GC to determine conversion of 1-decene to undesired byproducts. Test B: A similar experiment was conducted under the same conditions without the addition of 2-ethylhexanoic acid. In Test B, more than 10% of the 1-decene stream was converted to branched compounds, dienes, and paraffins, with the primary pathway being isomerization to an internal olefin. In the presence of the deactivating agent (Test A), no conversion of 1-decene into undesired by-products was observed.

### Example 2

Test A: MMAO (7 wt % Al in heptane) was added to a flask and diluted with a solution of 67 wt % 1-octene (C8 stream) in heptane so that the [Al]=500 ppmw. 3 molar equivalents of the deactivating agent (2-ethylhexanoic acid in this example) were slowly added to the mixture while stirring. After gas evolution was no longer observed, a solution of 0.25 wt % iron catalyst (iron duroct+Ligand A, 1:1.9 molar ratio) in heptane was added to the mixture. The mixture was transferred to a stainless-steel autoclave with a stir bar and sealed within the glovebox. The vessel was removed from the glovebox and heated to 204° C. for 2-4 hours. Periodically, aliquots were removed from the reaction vessel, cooled, and analyzed by GC to determine conversion of 1-octene to undesired byproducts. Test B: A similar experiment was conducted under the same conditions without the addition of 2-ethylhexanoic acid. In Test B, greater than 10% of the 1-octene stream was converted to branched compounds, dienes, and paraffins, with the primary pathway being isomerization to an internal olefin. In the presence of the deactivating agent (Test A), no conversion of 1-octene into undesired by-products was observed.

### Example 3 (Excel)

Test A: In a glovebox, MMAO (7 wt % Al in heptane) was added to a flask and diluted with a 50 wt % solution of 1-decene in heptane so that the [Al]=500 ppmw. The mixture was stirred and heated to 95° C. and then 3.4 molar equivalents of 2-ethylhexanoic acid was added. After stirring for 5 minutes, the iron catalyst (iron duroct+Ligand A, 1:1.5 molar ratio) was added as a solid and the mixture was stirred for 30 minutes at 95° C. An addition funnel was attached to the flask and then the reaction apparatus was removed from the glovebox and put under an argon purge. A degassed, 0.1 M NaOH solution (1:1 volume with the reaction mixture) was charged to the addition funnel and then was slowly added to the reaction mixture at 95° C. After complete addition, the mixture was stirred at temperature for 15 minutes. Then, the stirring was stopped and the layers were allowed to fully separate (approximately 5 minutes). Aliquots were removed from both layers and analyzed to determine the amount of 2-ethylhexanoic acid in each layer. Test B: A similar experiment was conducted under the same conditions except using 2-ethyhexanol as the deactivating agent instead of 2-ethyhexanoic acid. The time for full separation using 2-ethylhexanol took longer (approx. 1 hour) compared to the carboxylic acid. The results of the experiments indicate that 2-ethylhexanoic acid is preferred as the deactivating agent because it more readily partitions into the aqueous phase compared to the alcohol deactivating agent.

TABLE 1

| | | Deactivating agent |
|---|---|---|
| Deactivating Agent | Phase | distribution (ppmw) |
| 2-ethylhexanoic acid | Organic | 0.3 |
| | Aqueous | 6605 |
| 2-ethyl-1-hexanol | Organic | 7228 |
| | Aqueous | N/A |

1. A process for producing alpha-olefins comprising:
a. contacting an ethylene feed with an oligomerization catalyst system, the catalyst system comprising a metal-ligand catalyst and a co-catalyst, in an oligomerization reaction zone under oligomerization conditions to produce a product stream comprising alpha-olefins;
b. withdrawing the product stream from the oligomerization reaction zone wherein the product stream further comprises oligomerization catalyst system;
c. contacting the product stream with a catalyst deactivating agent to form a deactivated product stream that contains deactivated catalyst components; and
d. heating the deactivated product stream to separate one or more components from the deactivated product stream.

2. The process of claim 1 wherein the metal is iron and the co-catalyst is modified methyl aluminoxane (MMAO).

3. The process of claim 1 wherein the product stream is not heated before step c).

4. The process of claim 1 wherein the temperature of the deactivated product stream at the end of step c) is no more than 10° C. higher than the temperature of the product stream from step b).

5. The process of claim 1 wherein the deactivated product stream is separated into a plurality of components in one or more separation steps.

6. The process of claim 5 wherein at least one of the separation steps produces a bottoms stream comprising deactivated catalyst components.

7. The process of claim 1 further comprising a separation step wherein the deactivated catalyst components are separated from a portion of the deactivated product stream.

8. The process of claim 7 where the separation step comprises contacting the deactivated product stream with an aqueous base stream.

9. The process of claim 1 wherein the catalyst deactivating agent comprises a carboxylic acid.

10. The process of claim 1 wherein the catalyst deactivating agent has a boiling point of at least 170° C.

11. The process of claim 1 wherein the catalyst deactivating agent has a boiling point of from 180 to 250° C.

12. The process of claim 1 wherein the catalyst deactivating agent comprises 2-ethylhexanoic acid.

13. The process of claim 1 wherein the catalyst deactivating agent comprises one or more esters.

14. The process of claim 1 wherein the catalyst deactivating agent comprises methyl acetoacetate.

15. The process of claim 1 wherein the aqueous base comprises sodium hydroxide.
